(19) United States
(12) Patent Application Publication
(10) Pub. No.: US 2015/0190621 A1
(43) Pub. Date: Jul. 9, 2015

(54) Device and Method for Tissue Displacement in Brachytherapy

(71) Applicant: UNIVERSITY OF FLORIDA RESEARCH FOUNDATION, INCORPORATED, Gainesville, FL (US)

(72) Inventors: Anamaria Reyna Yeung, Gainesville, FL (US); Richard David Helmig, Hawthorne, FL (US)

(21) Appl. No.: 14/427,384

(22) PCT Filed: Sep. 10, 2013
(86) PCT No.: PCT/US2013/058895

§ 371 Date: Mar. 11, 2015

Related U.S. Application Data
(60) Provisional application No. 61/699,535, filed on Sep. 11, 2012.

(51) Int. Cl. A61M 29/02; A61N 5/10

(52) U.S. Cl. CPC A61M 29/02; A61N 5/1016; A61B 2018/0022

(57) ABSTRACT

The present invention provides a device for displacing healthy or normal tissue away from radiation exposure during brachytherapy. In one embodiment, the device of the present invention comprises an inflatable component attached to the ring of a tandem and ring brachytherapy applicator. When the tandem and ring applicator is inserted, the balloon is deflated so that patient discomfort is minimized. Once the ring is in the correct position, the balloon is inflated and the rectum is displaced. The balloon remains inflated during any image guidance that is performed and for the duration of the brachytherapy treatment. The balloon is deflated prior to the removal of the ring from the body.

### BACKGROUND OF INVENTION

Brachytherapy is a type of internal radiotherapy that involves placing a radiation source close to, or inside, a tissue that is to receive radiation treatment. Brachytherapy is generally regarded as an essential component of definitive radiotherapy for cervical cancer. In brachytherapy for cervical cancer, it is important to apply sufficient radiation to the target tumor tissues in the cervix, while minimizing exposure to adjacent healthy or normal tissues and organs such as the rectum and the bladder.

Various types of brachytherapy applicators have been developed. A common applicator for high dose rate (HDR) brachytherapy for cervical cancer is the tandem and ring applicator, which comprises round hollow holders (that allow the radiation source to dwell and pass through) that are placed adjacent to the cervix.

Currently, unwanted radiation exposure to the rectum during brachytherapy is reduced by extending the distance between the rectum and the radiation source-dwell position by the vaginal insertion of a separate bulky instrument (commonly referred to as a “rectal blade”) that has a “plastic spatula”-liked shape. The “rectal blade” is placed against the ring applicator, thereby physically displacing the rectum away from the radiation source.

However, the insertion of a bulky “rectal blade” as an additional component into the vagina can cause discomfort and pain in patients. Therefore, an alternative design for displacement of healthy or normal tissues during brachytherapy is needed.

### BRIEF SUMMARY

The present invention provides a brachytherapy device having a novel means for displacing healthy or normal tissue of a patient away from a radiation source-dwell position located inside a compartment of the brachytherapy device.

In one embodiment, the present invention provides a gynecological brachytherapy system comprising a tandem and ring brachytherapy applicator having at least one inflatable component (such a balloon), wherein the inflatable component is attached to, or mounted on, the ring of the tandem and ring brachytherapy applicator.

In one embodiment, the inflatable component of the present invention comprises at least one segment of inflatable tubing that is attached to the ring of a tandem and ring applicator. The tubing can be inflated and deflated in a controlled manner.

As the tandem and ring applicator is inserted into a patient, the inflatable component is deflated so that patient discomfort is minimized. In the deflated configuration the size of the device that is being inserted into the patient is roughly the same as it would be if the inflatable component were not present.

Once the ring is in the correct position in the cervix, the inflatable component is inflated thereby displacing the rectum. The inflatable component can be inflated with an appropriate fluid. In one embodiment, the inflatable component is inflated with a contrast agent to enhance the visualization of the device from its surrounding tissue. Appropriate imaging modalities can be utilized to facilitate the implant procedure.

The inflatable component can remain inflated during any imaging that is performed and for the duration of the brachytherapy treatment. The inflatable component is deflated prior to the removal of the ring from the vagina.

The subject invention further provides methods for using the inflatable device. The use of this device improves patient comfort and helps to reduce radiation exposure to non-target tissues.

### BRIEF DESCRIPTION OF DRAWINGS

FIG. 1 shows one embodiment of the device of the present invention.

FIG. 2 shows one embodiment of the inflatable component of the present invention.

### DETAILED DISCLOSURE

Brachytherapy is a type of internal radiotherapy that involves placing a radiation source close to tissue requiring radiation treatment. Brachytherapy is generally regarded as an essential component of definitive radiotherapy for cervical cancer.

Currently, radiation exposure to non-target tissue, such as the rectum, during brachytherapy is reduced by inserting, into the vagina, a separate bulky, plastic spatula” like instrument (commonly referred to as a “rectal blade”). Placing the “rectal blade” near where the radioactive source is located during the brachytherapy treatment, thereby physically displacing the rectum away from the radioactive source. However, the insertion of a bulky “rectal blade” as an additional component into the vagina causes discomfort in patients. Therefore, an improved design for brachytherapy applicators is needed.

The present invention improves upon the existing devices by achieving rectal displacement without the discomfort of the rectal blade. In one embodiment, the device of the present invention comprises an inflatable component (e.g. a balloon) that is attached to (or an integral part of) the ring of a tandem and ring applicator that is used for high dose rate (HDR) brachytherapy for cervix cancer.

In one embodiment, the inflatable component of the device of the present invention comprises at least one segment of tubing that is attached to the ring of the tandem and ring applicator. As the tandem and ring applicator is inserted into the patient, the tubing remains deflated status so that patient discomfort is minimized. Once the ring is in the correct position in the vagina, the tubing is inflated thereby increasing the distance between the radioactive source and non-target tissue (such as the rectum). The tubing can remain inflated during any imaging that is performed and for the duration of the brachytherapy treatment. The tubing is deflated prior to the removal of the ring from the vagina.

In one embodiment, the inflatable component comprises multiple inflatable portions, wherein at least one inflatable portion can transition, in a controlled manner, between an original, deflated, configuration and one or more inflated configurations.

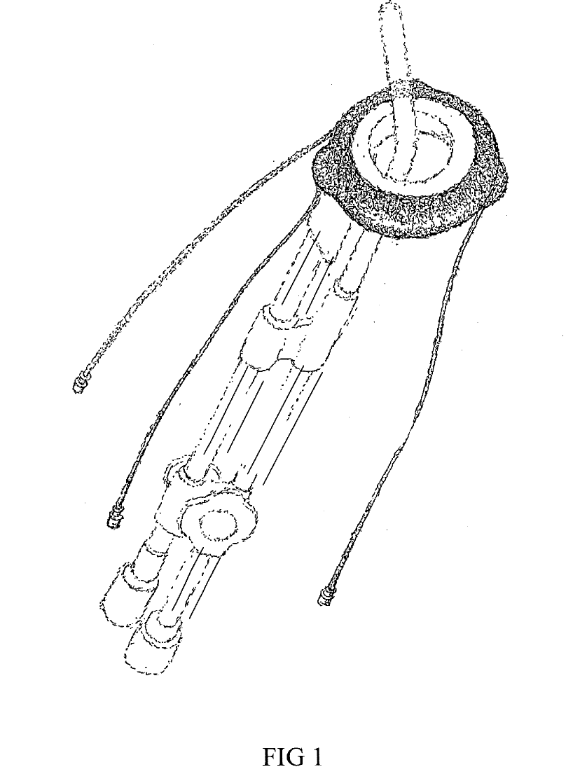

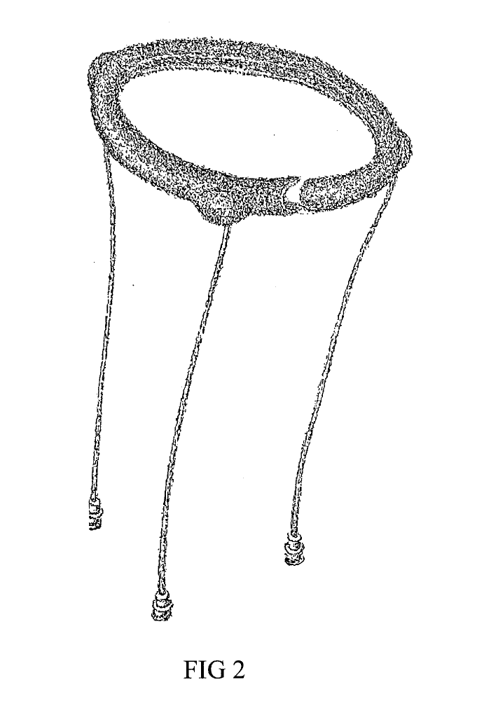

FIG. 1 shows one embodiment of the device of the present invention, wherein the inflatable tubing is attached to the ring of the tandem and ring gynecological brachytherapy applicator. FIG. 2 shows an embodiment of the inflatable component of the present invention.

### The Inflatable Component

The inflatable component of the subject invention, an example of which is shown in FIG. 2, can comprise an inner surface and an outer surface, wherein the inner surface is attached to, or mounted on, the ring of the tandem and ring brachytherapy applicator.

In one embodiment, the inflatable component comprises a plurality of inflatable portions, wherein each inflatable portion can transition, in a controlled manner, between an original, deflated configuration to one, or a plurality, of inflated configurations.

The inflatable component for tissue displacement can be of any suitable size, geometry, or configuration. In its inflated configuration, the inflatable device may have a shape including, but not limited to, a circular cylinder. The inflatable component, when inflated, can be capable of displacing tissue up to, for example, about 0.1, 0.2, 0.3, 0.4, 0.5, 0.6, 0.7, 0.8, 0.9, 1, 1.5, 2, 2.5, 3, 3.5, or 4 cm.

In one embodiment, the compartment of the brachytherapy applicator that encloses a radiation source during brachytherapy (e.g. the ring) is substantially, or entirely, surrounded by the inner surface of the inflatable component. The inner surface of the inflatable component can be attached to, or mounted on, the compartment of the brachytherapy applicator holding confining the radiation source during brachytherapy.

The inflatable component can be made of materials such as polyimide (kapton), polyester, fluoropolymers, natural and synthetic rubber (such as silicone rubber), silicone, thermoplastic elastomers, nylon, mylar, polyethelene (HDPE including radiated HDPE), latex, polyvinyl chloride. Other elastic materials known in the art may also be used for the inflatable component of the device of the present invention.

The inflatable component may be inflated by gas or liquid. In one embodiment, the inflatable component and/or the inflation fluid comprise a detectable material such as a contrast agent (such as a radiographically detectable material), for visualizing the position of the device when compared to the surrounding tissue/organs and/or the degree of inflation.

An imaging instrument (such as, x-ray, CT, and MRI) may be utilized for visualizing the insertion of the brachytherapy device, with the inflatable component of the present invention into the subject.

In one embodiment, the inflatable component comprises, or is in fluid communication with, at least one lumen that provides a conduit through which the fluid passes that will inflate an inflatable portion of the inflatable component.

The inflation fluid (e.g. contrast agent) can be delivered into the inflation lumen using, for example, a syringe. The fluid will pass through the inflation lumen into the inflatable component thereby causing the flexible walls of the inflatable component to expand (inflate). Upon completion of the procedure (or when it is no longer necessary to displace tissue via inflation of the inflatable component), the inflation fluid can be removed from the inflatable component through the inflation lumen again using, for example, a syringe.

### Devices

The inflatable component can be adapted for attachment to, for example, a tandem and ring brachytherapy applicator and an ovoid brachytherapy applicator. Commercially-available tandem and ring brachytherapy applicators include, but are not limited to, Varian® Varisource™ Brachytherapy Applicator AL13017000, and NUCLETRON™ brachytherapy applicators

In one embodiment, the brachytherapy applicator (such as the tandem and ring applicator) of the subject invention comprises:

a first member for confining a radiation source during brachytherapy; and

a second member comprising an open proximal end, an elongated hollow body, and a distal end, wherein the second member is connected to the first member and allows for removable insertion of a radiation source into the open proximal end, through the hollow elongated body, to the first member.

The term “distal end,” as used herein, refers to the end of the brachytherapy applicator adjacent to target tissue of the patient receiving radiation treatment, when the applicator is inserted into the patient's body. The term “proximal end,” as used herein, refers to the end of the brachytherapy applicator projecting external of the patient's body during brachytherapy, and is connected to an HDR machine through transfer tubes.

As used herein “connected” or “attached” includes physical, whether direct or indirect, permanently affixed or adjustably mounted, such as for example, the inflatable component for tissue displacement is mounted on the compartment of the applicator holding the radiation source (such as the ring of the ring and tandem applicator).

In one embodiment of the tandem and ring applicator, the first member is a ring. In one embodiment, the inner surface of the inflatable body is attached to the outer surface of the ring of the tandem and ring applicator. In one embodiment of the tandem and ring applicator, the elongated body of the second member is cylindrical, such as a hollow tube. The hollow tube of the brachytherapy applicator allows passage through which a radiation source may be advanced.

The radiation sources that may be used with the brachytherapy applicators include sealed radiation sources in a form of pellets, capsules, rods, needles, wires, and the like. The radiation source may include, but are not limited to, radionuclides such as cesium, and iridium.

### Applications

The inflatable component can be adapted for use with brachytherapy applicators of various types including, but not limited to, intracavity and intraluminal applicators. The inflatable device for tissue displacement can be used with a brachytherapy applicator for delivery of radiation treatment to tissues and organs including, but not limited to, the prostate, breast, skin, bladder, pelvis, rectum, esophagus, trachea, oral, nasopharynx, oropharynx, head, neck, coronary and vascular tissues, the thoracic cavity, pleural space, and retroperitoneal space.

In one embodiment, the inflatable component for tissue displacement during brachytherapy is adapted for use with a gynecological brachytherapy applicator. In certain embodiments, the gynecological brachytherapy applicator is used in the delivery of radiation to tissues and organs including, but not limited to, the cervix, paracervical area, cervical channel, vagina, vaginal cuff, ovary, uterus, and endometrium. The inflatable component can be used with a brachytherapy applicator in the treatment of gynecologic cancers such as cervical cancer, endometrial cancer, uterine cancer, ovarian cancer, and vaginal cancer.

In one embodiment, the inflatable device is adapted for coupling with an applicator in low dose rate (LDR) and/or high dose rate (HDR) brachytherapy. Brachytherapy is divided into two major classifications, low dose rate (LDR) and high dose rate (HDR). Low dose rate treatments typically involve dose rates of 40 to 60 centigray per hour and more typically about 55 centigray/hr. High dose brachytherapy contemplates significantly higher rates of dose delivery (up to 1000 centigray per minute) and not uncommonly in the range of hundreds of centigray in minutes.

### Methods of Use

During radiation therapy, the radiation source (and the applicator compartment enclosing the radiation source) is positioned in close proximity to target tissues for receiving radiation treatment. However, healthy tissue may also be in close proximity to the radiation source.

The present invention provides a method for displacing healthy or normal tissue of a subject away from a radiation source that is dwelled inside a compartment of the applicator. In a specific embodiment, the method comprises:

inserting the brachytherapy applicator, attached to an inflatable device of the present invention, into a patient;

positioning the brachytherapy applicator into a suitable position inside the patient body, such as positioning the compartment adjacent to, target tissue receiving radiation treatment, wherein the inflatable remains its deflated state during the insertion and positioning of the brachytherapy applicator;

when the brachytherapy applicator is positioned into a suitable position, inflating one or more inflatable portions of the inflatable device;

applying brachytherapy to the patient; and before removing the brachytherapy applicator from the patient, deflating the inflatable device.

When the brachytherapy applicator is inserted into a subject, the inflatable device remains deflated so that patient discomfort is minimized. Once the applicator is in the correct position in the patient, the inflatable device is inflated and the healthy or normal tissue is displaced. The inflatable device can remain inflated for the duration of the brachytherapy treatment, and is deflated prior to the removal of the brachytherapy applicator from the patient.

The term “patient” or “subject,” as used herein, describes a mammal. Mammalian species that can benefit from the current invention include, but are not limited to, primates such as apes, chimpanzees, orangutans, humans, monkeys; and other animals such as dogs, cats, horses, cattle, pigs, sheep, goats, chickens, mice, rats, guinea pigs, and hamsters. In one embodiment, the patient is a human. In another embodiment, the patient is a non-human mammal.

Following is an example that illustrates procedures and embodiments for practicing the invention. The examples should not be construed as limiting.

### Example 1

### Brachytherapy Applicator with Inflatable Component

In one embodiment, the present invention provides a device having an inflatable component for displacing healthy or normal tissue of a subject away from a radiation source situated inside a compartment of a brachytherapy applicator, wherein the inflatable device comprises:

an inflatable component comprising one or more inflatable portions, wherein at least one inflatable portion can transition, in a controlled manner, between an original, deflated configuration and one or more inflated configurations; and

at least one lumen for conducting a fluid for inflating or deflating an inflatable portion on the inflatable component, wherein the lumen is functionally connected (e.g. in fluid communication with) to the inflatable component;

wherein the inflatable component comprises an inner surface and an outer surface, and the inner surface has a shape conformable to the shape of the ring of a ring and tamdem brachytherapy applicator.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. An inflatable device for displacing healthy or normal tissue of a subject away from a radiation source situated inside a compartment of a brachytherapy applicator, wherein the inflatable device comprises:
an inflatable body comprising one or more inflatable portions, wherein at least one inflatable portion is movable between an original, deflated configuration and one or more inflated configurations; and
at least one member for inflating or deflating an inflatable portion on the inflatable body, wherein the member is functionally connected to the inflatable body;
wherein the inflatable body comprises an inner surface and an outer surface, and the inner surface has a shape conformable to the shape of the brachytherapy applicator compartment enclosing a radiation source during brachytherapy treatment.

2. The inflatable device, according to claim 1, wherein the inner surface of the inflatable body substantially surrounds the brachytherapy applicator compartment that encloses a radiation source during brachytherapy.

3. The inflatable device, according to claim 1, wherein the inflatable body is cylindrical.

4. The inflatable device, according to claim 3, wherein the inflatable body has a ring structure.

5. The inflatable device, according to claim 1, wherein the member for inflating or deflating an inflatable portion on the inflatable body is a lumen or a hollow tube.

6. The inflatable device, according to claim 1, wherein the member for inflating or deflating an inflatable portion on the inflatable body is connected to the inflation body and is capable of supplying inflation material to the inflatable body.

7. The inflatable device, according to claim 1, wherein the inflation material is inflation gas or inflation fluid.

8. The inflatable device, according to claim 1, made of material comprising polyimide, polyester, fluoropolymers, rubber, silicone, thermoplastic elastomer, nylon, mylar, polyethelene, latex, polyvinyl chloride, or a combination thereof.

9. The inflatable device, according to claim 1, made of material comprising rubber.

10. A brachytherapy applicator apparatus comprising: an inflatable device of claim 1, and a brachytherapy applicator.

11. The brachytherapy applicator apparatus according to claim 10, wherein the brachytherapy applicator is a tandem and ring applicator.

12. A method for displacing healthy or normal tissue of a subject away from a radiation source situated inside a compartment of a brachytherapy applicator, wherein the method comprises:
inserting a brachytherapy applicator, attached to an inflatable device of claim 1, into a patient;
positioning the brachytherapy applicator into a suitable position inside the patient body, wherein the inflatable device is in its deflated state during the insertion and positioning of the brachytherapy applicator;
when the brachytherapy applicator is positioned into a suitable position, inflating one or more inflatable portions of the inflatable device;
applying brachytherapy to the patient; and
before removing the brachytherapy applicator from the patient, deflating the inflatable device.

13. The method, according to claim 12, wherein the brachytherapy applicator is a tandem and ring applicator.

14. The method, according to claim 12, wherein the inner surface of the inflatable body substantially surrounds the brachytherapy applicator compartment that encloses a radiation source during brachytherapy.

15. The inflatable device, according to claim 12, wherein the inflatable body is cylindrical.

16. The inflatable device, according to claim 15, wherein the inflatable body has a ring structure.

17. The inflatable device, according to claim 12, wherein the member for inflating or deflating an inflatable portion on the inflatable body is a lumen or a hollow tube.

18. The inflatable device, according to claim 12, wherein the member for inflating or deflating an inflatable portion on the inflatable body is connected to the inflation body and is capable of supplying inflation material to the inflatable body.

19. The inflatable device, according to claim 12, wherein the inflation material is inflation gas or inflation fluid.

20. The inflatable device, according to claim 12, wherein the inflatable device is made of material comprising polyimide, polyester, fluoropolymers, rubber, silicone, thermoplastic elastomer, nylon, mylar, polyethelene, latex, polyvinyl chloride, or a combination thereof.
